(19) United States
(12) Patent Application Publication
(10) Pub. No.: US 2025/0177048 A1
(43) Pub. Date: Jun. 5, 2025

(54) STEREOTACTIC PROCEDURE PLANNING FOR ROBOTIC SURGICAL PROCEDURES

(71) Applicant: Baylor College of Medicine, Houston, TX (US)

(72) Inventors: Jeffrey Treiber, Houston, TX (US); Daniel Curry, Houston, TX (US)

(73) Assignee: Baylor College of Medicine, Houston, TX (US)

(21) Appl. No.: 18/939,178

(22) Filed: Nov. 6, 2024

Related U.S. Application Data
(60) Provisional application No. 63/605,762, filed on Dec. 4, 2023.

(51) Int. Cl. A61B 34/10; A61B 34/30; G16H 30/40; G16H 50/70

(52) U.S. Cl. CPC A61B 34/10; A61B 34/30; G16H 30/40; G16H 50/70; A61B 2034/105; A61B 2034/107; A61B 2034/305

(57) ABSTRACT

Systems, methods, and devices for planning stereotactic surgical procedures are disclosed. In one aspect, a method includes: accessing surgical plans for previous stereotactic procedures performed on a group of patients, the surgical plans including imaging data representing an anatomical feature of each patient in the group; applying one or more transforms to register the imaging data for each patient to a template of the anatomical feature for the group of patients; generating one or more density maps for a trajectory targeting at least one region of interest (ROI) in the anatomical feature, based on the registered imaging data; and generating a surgical plan to perform a current stereotactic procedure on a subject patient, based on the one or more density maps, the surgical plan including the trajectory targeting the at least one ROI in the anatomical feature of the subject patient. Other aspects and features are also claimed and described.

This application claims priority to U.S. Provisional Application No. 63/605,762 filed Dec. 4, 2023, which is incorporated herein by reference in its entirety.

### FIELD OF THE DISCLOSURE

The instant disclosure relates to planning stereotactic surgical procedures, and particularly, in some embodiments, to planning trajectories for stereotactic neurosurgical procedures.

### BACKGROUND

Stereotactic neurosurgery is a rapidly growing field that utilizes minimally invasive techniques to access the brain with minimal approach-related morbidity. Conventional neurosurgical techniques rely on visualizing the target areas directly by retracting and sometimes removing normal brain tissue. Modern stereotactic neurosurgical procedures, however, involve making small incisions and small holes in the skull to accurately pass small instruments, such as a needle or electrode into the brain using a map of the patient's anatomy created from their medical imaging. This is usually done by selecting a target point for treatment or sampling and a trajectory to reach that target with minimal injury to normal brain structures. These stereotactic plans are usually created prior to the surgical procedure.

Planning such stereotactic procedures, however, is a complex and time-consuming process that increases in complexity as the number of stereotactic trajectories and targets grows. Creating an optimal surgical plan requires not only having a thorough understanding of general neuroanatomy and safe entry points for stereotactic trajectories but also knowing how to individualize the plan for the patient's specific neuroanatomy and surgical indication. Inaccuracies in a stereotactic surgical plan can put a patient at risk for severe complications or even death.

### SUMMARY

Embodiments of this disclosure may be used to provide a surgery planning tool for stereotactic procedures. Such a planning tool may use existing surgical plans from previous stereotactic procedure performed on a group of patients to create a surgical plan for a current stereotactic procedure (e.g., a neurosurgical stereotactic procedure) to be performed on a subject patient. In some embodiments, imaging data (e.g., CT scans and MRI scans) representing an anatomical feature (e.g., a brain) of each patient in the group may be extracted from the existing surgical plans and processed for further analysis. The imaging data may include information, such as spatial coordinates, for one or more trajectories targeting one or more anatomical regions of each patient. The imaging data may be processed by registering the imaging data to a standard anatomical template (e.g., an average brain template for the group of patients) and applying one or more transforms to the spatial coordinates of the existing surgical plans. The registered imaging data may then be used to generate a density map of the entry and target points used to target a specific anatomical region. The density map may then be used to generate a surgical plan for performing the current stereotactic procedure.

According to one embodiment, a method of creating surgical plans for stereotactic procedures includes: accessing surgical plans for previous stereotactic procedures performed on a group of patients, the surgical plans including imaging data representing an anatomical feature of each patient in the group; applying one or more transforms to register the imaging data for each patient to a template of the anatomical feature for the group of patients; generating one or more density maps for a trajectory targeting at least one region of interest (ROI) in the anatomical feature, based on the registered imaging data; and generating a surgical plan to perform a current stereotactic procedure on a subject patient, based on the one or more density maps, the surgical plan including the trajectory targeting the at least one ROI in the anatomical feature of the subject patient.

In some embodiments, the surgical plans may further include information identifying a plurality of targeted regions in the anatomical feature for the group of patients and coordinates of the trajectory points for each targeted region in a three-dimensional (3D) space. The at least one ROI may be selected from among the plurality of targeted regions. The trajectory targeting the ROI may include an entry point and a target point for each targeted region. The one or more transforms applied to register the imaging data for each patient in the group to the template may include a first transform calculated to register the imaging data for each patient to the template of the anatomical feature. The first transform may then be applied to register the coordinates of the respective entry and target points for each targeted region from a first native space associated with each patient in the group to a template space associated with the template of the anatomical feature. The one or more density maps for each targeted region may include distributions of the corresponding entry and target points in the template space. Generating the density map(s) may include calculating the coordinates of the respective entry and target points for each targeted region in the template space based on the corresponding distributions. The surgical plan for the subject patient may be generated by first calculating a second transform to register an image of the anatomical feature of the subject patient to the template of the anatomical feature. An inverse of the second transform may be applied to map the registered coordinates of the respective entry and target points for the at least one ROI to the anatomical feature of the subject patient in a second native space. The surgical plan for the current stereotactic procedure on the subject patient may then be generated, based on the entry and target points for at least one ROI mapped to the anatomical feature of the subject patient in the second native space.

In some implementations, the method may be embedded in a computer-readable medium as computer program code comprising instructions that cause a processor to perform operations corresponding to the steps of the method. In some embodiments, the processor may be part of a surgical robot system including a surgical tool and a processor coupled to the first network adaptor, and the memory. The surgical robot system (or surgical robot) may include a robotic arm with a robotic surgical tool that is used to perform the current stereotactic procedure on the subject patient, for example, by controlling the robotic arm according to the surgical plan generated using the disclosed stereotactic planning techniques. In some embodiments, the one or more density maps may include a distribution of each trajectory point associated with the trajectory of the robotic arm in a three-dimensional (3D) template space representing the anatomical feature, and a position of the robotic arm may be controlled during the current stereotactic procedure according to the distribution of each trajectory point associated with the trajectory of the robotic arm in the 3D template space. Accordingly, the disclosed surgical planning techniques may be used to produce surgical decision-making trajectory maps that can be used as templates for standardizing safety and workflow improvements. Such templates have the potential to reduce surgeon fatigue, standardize trajectory planning, provide more accurate trajectories for improved placement of surgical tools during stereotactic procedures, increase accessibility, decrease costs, and ultimately lead to improved patient care with fewer patient deaths.

As used herein, the term “coupled” means connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially parallel includes parallel), as understood by a person of ordinary skill in the art.

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus or system that “comprises,” “has,” or “includes” one or more elements possesses those one or more elements, but is not limited to possessing only those elements. Likewise, a method that “comprises,” “has,” or “includes,” one or more steps possesses those one or more steps, but is not limited to possessing only those one or more steps.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is a diagram illustrating an overhead view of a surgical operating room including a surgical robot for performing various neurosurgical stereotactic procedures according to some embodiments of the disclosure.

FIG. 2 is a diagram of a density heat map showing the distributions of entry and target points for existing trajectories to target two different regions of the insula overlaid onto a template brain according to some embodiments of the disclosure.

FIG. 3 is a flow chart of an example method of creating surgical plans for stereotactic procedures according to some embodiments of the disclosure.

FIG. 4 is a diagram of an example anatomical template of a brain representing an average of the brain images for a group of patients according to some embodiments of the disclosure.

FIG. 5 is an example data flow for calculating density maps for entry and target points for a specific region of interest (ROI) according to some embodiments of the disclosure.

FIG. 6A is diagram of an example trajectory generated between an entry point and a target point mapped to an anatomical template of a human brain in a three-dimensional (3D) space according to some embodiments of the disclosure.

FIG. 6B is a diagram of the anatomical template showing distributions of the entry point and the target point mapped to the anatomical template in the 3D space according to some embodiments of the disclosure.

FIG. 7 is an example data flow for generating a surgical plan to perform stereotactic neurosurgery on a subject patient based on density maps of entry and target points for an ROI in the patient's brain according to some embodiments of the disclosure.

FIG. 8 is a block diagram of an example computer system in which embodiments of the disclosure may be implemented.

### DETAILED DESCRIPTION

Embodiments of this disclosure may be used to provide a surgery planning tool for stereotactic procedures. Such a tool may be used to apply surgical principles in a fast, accurate, and unbiased way to create surgical plans with trajectories that a surgeon can review and modify as needed based on patient-specific information prior to or during surgery. The applied principles may be based on considerations of relevant anatomical structures and classifications, which vary across medical and research disciplines. For example, the relevant anatomy of a patient undergoing stereotactic neurosurgery may include a region of interest inside the patient's brain as well as all the tissue that a stereotactic instrument must pass through as it travels along a trajectory between an entry point on the patient's skull and a target point corresponding to the region. Considerations of bone thickness, trajectory angle, and lesion coverage as well as the locations of cerebrospinal fluid spaces, blood vessels, and eloquent brain areas can influence a neurosurgeon's surgical plan. These considerations may be determined based on pre-operative imaging data acquired using any of various medical imaging techniques.

In some embodiments, patient data (e.g., computerized tomography (CT) scans, magnetic resonance imaging (MRI) scans, and/or EEG recordings) and other relevant information from existing surgical plans for a stereotactic procedure previously performed on a group of patients may be used to generate a surgical plan for a new patient subject to the procedure. In some implementations, the information from the previous plans may be categorized by target name (or region), coordinates of the target, and coordinates of the entry point for each trajectory of the surgical tool in the previously performed stereotactic surgical plans. The imaging data and coordinates from the plans may be registered to a standard anatomical template, and one or more density maps of the target and entry points for each surgical objective may be created. Such a “density map” may represent the number of times that an entry point or a target point for a region of interest (ROI) occurs at a spatial coordinate within the existing surgical plans for the group of patients divided by the total number of patients with this same ROI included in their respective surgical plans. For various applications, these density maps may be generated by the exact coordinates or from clusters of nearby points, such as placing a sphere of an arbitrary size at the entry or target point prior to performing the density calculation. The one or more maps may then be used to generate an initial surgical plan that can be further modified based on patient-specific information.

The initial plan may be generated with trajectories based on the surgical principles and considerations that are relevant to the procedure, as described above. For example, the surgical plan generated for the neurosurgical procedure described above may include intracranial stereotactic trajectories that avoid collisions with other trajectories, remain orthogonal to the outer table of the patient's skull, avoid vascular structures and healthy tissues, minimize trajectory distance, and use supratentorial entry points when possible. The surgical plan may be utilized by robotic, frame-based, or other stereotactic surgical systems during neurosurgery to map pre-operative and/or intra-operative imaging data acquired for the subject patient to the actual anatomical structures in a surgical environment surrounding each trajectory targeting a region of interest (ROI) within the patient's brain. The disclosed surgical planning techniques may be used to produce surgical decision-making trajectory maps that can be used as templates for standardizing safety and workflow improvements. Such templates have the potential to reduce surgeon fatigue, standardize trajectory planning, provide more accurate trajectories for improved placement of surgical tools during stereotactic procedures, increase accessibility, decrease costs, and ultimately lead to improved patient care with fewer patient deaths.

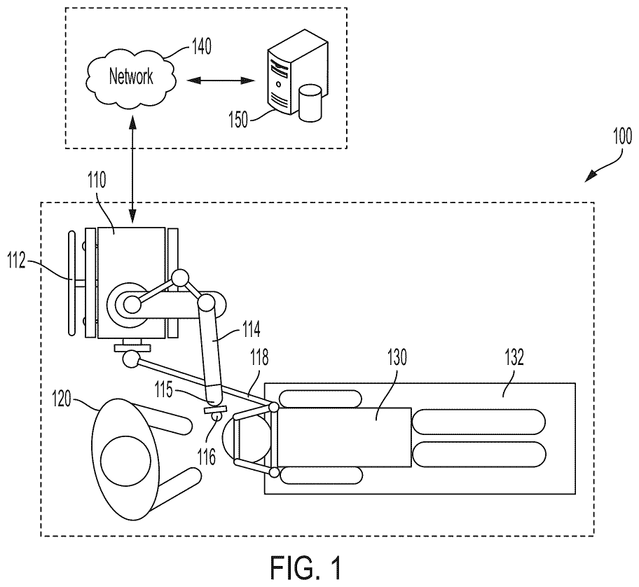

While the disclosed techniques will be described below with reference to FIGS. 1-7 in the context of neurosurgical stereotactic procedures, it should be appreciated that the disclosed techniques are not intended to be limited thereto and that these techniques may be applied to any of various stereotactic procedures. In some embodiments, the disclosed techniques may be implemented as part of a surgical planning software application executable at a computing device (e.g., a desktop or mobile computer). In some implementations, the surgical planning software may be integrated with a surgical robot system (or “surgical robot”) designed to assist a surgeon (e.g., a neurosurgeon) plan and perform a stereotactic procedure (e.g., stereotactic neurosurgery) on a patient, as will be described in further detail below with reference to FIG. 1.

For purposes of this disclosure, a surgical robot or robot system may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for stereotactic planning, guidance, imaging, or other purposes. In some embodiments, the surgical robot may include a processor (such as a central processing unit (CPU) or other computing circuitry having one or more processing cores) and a memory coupled to the processor. The memory may include, for example, a random access memory (RAM), a read-only memory (ROM), a hard disk drive (HDD), a solid state drive (SSD), and/or any other computer-readable storage medium used to store information (e.g., software instructions and data) in a persistent or non-persistent state. Additional components of the surgical robot may include one or more network ports for communicating with external devices as well as various input and output (I/O) devices. The input devices of the surgical robot may include, for example, a keyboard, a mouse, or a capacitive touchscreen integrated with a display. The surgical robot may also include one or more buses operable to transmit communications between the various hardware components. As will be described in further detail below, some of these components may be implemented as part of a surgical planning and guidance control system that enables a surgeon to plan, control, and coordinate actions of the surgical robot during the stereotactic procedure.

FIG. 1 is a diagram illustrating an overhead view of a surgical operating room 100 including a surgical robot 110 for performing various neurosurgical stereotactic procedures according to some embodiments of the disclosure. Examples of such procedures include, but are not limited to, stereoelectroencephalography (SEEG), deep brain stimulation (DBS), biopsy, and neuroendoscopy. Surgical robot 110 may be used, for example, to assist a surgeon 120 in performing such a stereotactic procedure on a patient 130. In some embodiments, surgical robot 110 may include a display 112, at least one robotic (or robot) arm 114, an end effector 115, a surgical instrument 116, and a stereotactic frame 118.

The stereotactic procedure in this example may be performed with stereotactic frame 118 placed around the head of patient 130. Stereotactic frame 118 may be secured to an operating table 132 to prevent movement of the patient 130's head during the procedure. As shown in FIG. 1, stereotactic frame 118 may include an auxiliary arm extending from a base of surgical robot 110 to operating table 132 to prevent the movement of patient 130 or head thereof relative to robot arm 114 or other components of surgical robot 110 during surgery. However, it should be appreciated that the disclosed stereotactic planning techniques are not intended to be limited to frame-based stereotactic procedures and that these techniques may be applied to frameless stereotactic procedures.

Although not shown in FIG. 1, it should be appreciated that surgical robot 110 may include any number of additional components or devices as desired for a particular implementation. It should also be appreciated that the components of surgical robot 110 may be arranged in any of various configurations or locations as desired or necessary for a particular surgical procedure and that embodiments of the present disclosure are not intended to be limited to the particular arrangement shown in FIG. 1. For example, while surgical robot 110 and robot arm is positioned above the head of patient 130 in the operating room 100 of FIG. 1, it should be appreciated that surgical robot 110 may be positioned at any suitable location near patient 130 depending on the type of surgical procedure being performed or area of the patient's body undergoing the procedure. Also, while display 112 is shown in FIG. 1 as being attached to or integrated with surgical robot 110, display 112 in other implementations may be a separate device located in the operating room 100 or in a remote location and communicatively coupled to surgical robot 110 (e.g., via a network 140).

In some implementations, display 112 may be part of a separate computing device (such as a surgical workstation) coupled to surgical robot 110 via a wired or wireless connection. The computing device may be located in the operating room 100 with surgical robot 110 or in a remote location. Examples of such a computing device include, but are not limited to, a workstation, a personal computer (e.g., a desktop or laptop computer), a tablet computer, a mobile device (e.g., personal digital assistant (PDA) or smart phone), a remote server, a network storage device, or any other suitable device and may vary in size, shape, performance, functionality, and price. The remote server may be, for example, a remote database server 150 communicatively coupled to surgical robot 110 via a network 140. Network 140 may include a wired network, a wireless network, or a combination thereof. For example, network 140 may include a Bluetooth personal area network (PAN), a local area network (LAN), a wireless LAN (WLAN), a wide area network (WAN) (e.g., the Internet), a cellular network (e.g., a 4G long term evolution (LTE) network, a 5G network, or a 6G network), a wired network, one or more other networks, or any combination thereof.

In some embodiments, display 112 may be used to present information and provide input options to assist surgeon 120 with planning and performing a stereotactic procedure. For example, the display 112 may be part of a control unit of the surgical robot 110 used for surgical planning and control of a robotic surgical tool along a planned trajectory during the stereotactic procedure. In some implementations, display 112 may be mounted to a frame or body of surgical robot 110 with an adjustable bracket or retractable mount that allows surgeon 120 to independently move and/or orient display 112 as desired during the procedure. Display 112 may be part of, for example, a user interface or human-machine interface (HMI) for surgeon 120 to input commands for controlling the spatial movement, position, and/or orientation of robot arm 114 and surgical instrument 116 during the procedure. Robot arm 114 may be movable along or about an axis relative to a base of surgical robot 110. Surgical instrument 116 may be any of various surgical tools held by end effector 115 attached to the end of robot arm 114. End effector 115 may include any of various devices designed to grip, lift, move, and manipulate surgical instrument 116 or other objects in the environment during the procedure. For example, end effector 115 may include cameras, sensors or other feedback mechanisms to provide information about a surgical site as well as mechanisms for precise actuation and control of surgical instrument 116 in response to the commands or other input received from surgeon 120 via the HMI of surgical robot 110.

In some implementations, surgeon 120 may interact with the HMI via a touchscreen integrated with display 112 or other input devices (e.g., hand controllers) to navigate the surgical site, accurately position robot arm 114, and perform precise movements and actions with surgical instrument 116. Display 112 may provide surgeon 120 with real-time visual feedback and guidance information to assist surgeon 120 during the procedure. Surgeon 120 may use display 112 to monitor a status of surgical robot 110, view live video feeds from one or more cameras (not shown) coupled to surgical robot 110, and access relevant patient data. The patient data may include, for example, pre-operative and/or intra-operative patient data (e.g., CT scans, MRI scans, and/or EEG recordings) that can be used to identify locations of critical structures and create a surgical plan with trajectories targeting one or more regions of interest in an anatomical feature (such as the brain) of patient 130.

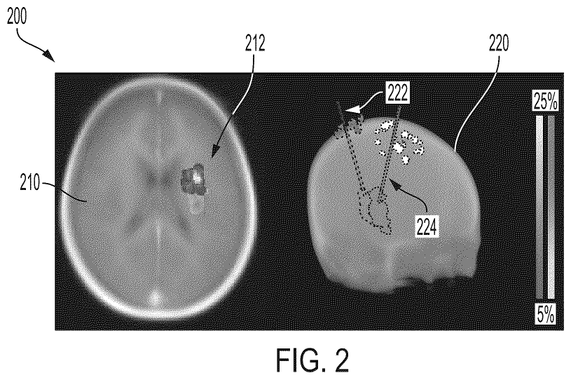

In some embodiments, the surgical plan may be generated by the surgical robot 110 (or control unit thereof) for a new patient (such as patient 130) based on the imaging data and other relevant information obtained from existing or previously performed stereotactic surgical plans associated with a group of patients. In some implementations, the existing surgical plans may be retrieved from a local data store or memory of surgical robot 110. Additionally or alternatively, the existing plans may be retrieved from a remote data store communicatively coupled to surgical robot 110 (e.g., from database server 150 via network 140). As described above, the previous plans may include information identifying at least one target (or ROI) in an anatomical feature (e.g., the brain) of each patient as well as coordinates of the respective entry and target points for each trajectory of a robotic surgical tool targeting the at least one ROI. The robotic surgical tool may be, for example, surgical instrument 116 held by end effector 115 attached to the end of robot arm 114 of surgical robot 110, as described above. The imaging data and coordinates from the existing plans may be registered to a standard anatomical template (e.g., an average brain template for the group of patients), and a density map of the target and entry points for one or more trajectories targeting each ROI may be created and visualized (e.g., via display 112) for surgeon 120 to review and modify as needed for the new patient. In some embodiments, the density map may be a two-dimensional (2D) or three-dimensional (3D) spatial and/or visual representation of the anatomical feature. An example of such a density map is shown in FIG. 2.

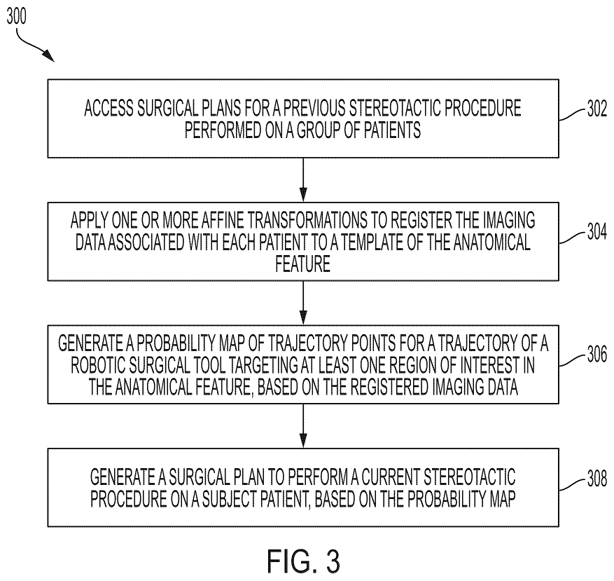

FIG. 2 is a diagram of a density map 200 showing the distributions of entry and target points of surgical trajectories used to target the insula within a template brain according to some embodiments of the disclosure. In this example, a left-hand portion of density map 200 shows a cross-sectional image (e.g., an MRI scan) of a template brain with visual cues indicating the locations of different insular regions 212. Insular regions 212 may include, for example, an insula short gyrus and an insula long gyrus within the cerebral cortex of the patient's brain. A right-hand portion of density map 200 shows a 3D trajectory map 220 that includes a trajectory 222 targeting the insular short gyrus and a trajectory 224 targeting the long insular gyrus. As described above and as will be described in further detail below with respect to FIG. 3, density map 200 (and other density maps) created from existing surgical plans for a stereotactic procedure previously performed on a group of patients may be used to generate a surgical plan to perform the stereotactic procedure on a new or subject patient (e.g., patient 130 of FIG. 1).

FIG. 3 is a flow chart of an example method 300 of creating surgical plans for stereotactic procedures according to some embodiments of the disclosure. As shown in FIG. 3, method 300 begins at block 302, which includes accessing surgical plans for a stereotactic procedure previously performed on a group of patients. In some embodiments, the surgical plans may include imaging data representing an anatomical feature (e.g., the brain) of each patient in the group. The stereotactic procedure may be, for example, a neurosurgical stereotactic procedure. Accordingly, the imaging data may include imaging data for each patient in the group of patients. At block 304, one or more transforms may be applied to register the imaging data for each patient to a template of the anatomical feature for the group of patients.

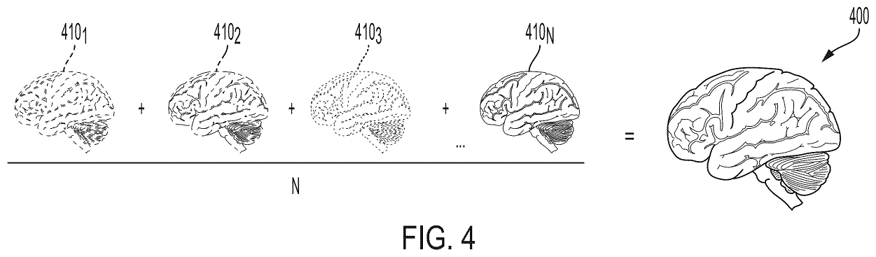

FIG. 4 is a diagram of an example anatomical template 400 of a human brain. In some embodiments, the anatomical template 400 may be represent an average of the brain images for a group of patients. For example, patient data (e.g., CT scans, MRI scans, and/or EEG recordings) may be obtained from a surgical plan associated with each patient in the group. As shown in FIG. 4, the imaging data may include a brain image 410 corresponding to each patient in a group of patients numbered 1 to N, where N may be any integer depending on the number of existing surgical plans or relevant patient imaging data that may be available for use. Accordingly, the anatomical template 400 in this example may be generated by taking an average of the brain images 4101 to 410N.

Returning to FIG. 3, method 300 proceeds to block 306, which includes calculating one or more density maps for a trajectory targeting one region of interest (ROI) in the anatomical feature, based on the imaging data registered to the template at block 304. As will be described in further detail below, the trajectory points may include an entry point and a target point for each trajectory of a surgical tool targeting at least one ROI. In some implementations, one or more machine learning models (e.g., a convolutional neural network (CNN) or other type of artificial neural network (ANN)) may be used to calculate the density maps and coordinates of the respective entry and target points for each ROI. At block 308, the density maps may be used to generate a surgical plan to perform the stereotactic procedure on a subject patient. The density maps may be adapted for any of various use cases and technologies, as desired for a particular implementation. For example, a surgeon may use the density maps in conjunction with surgical planning software executable at a computing device to select points within the density maps to define one or more trajectories for each ROI. In some implementations, the surgical planning software may be integrated with a surgical robot, e.g., surgical robot 110 of FIG. 1, as described above, used to perform stereotactic procedures and accessed by the surgeon via a human-machine interface (HMI) of the surgical robot. Additionally or alternatively, a preliminary trajectory may be defined as the maximum density of the respective entry and target points within the density maps calculated for each trajectory. The preliminary trajectory may then be reviewed by the surgeon and adjusted for patient specific features. In some embodiments, the density maps may be post-processed using any of various computer imaging and/or medical imaging techniques to further refine the preliminary trajectory. For example, such techniques may include, but are not limited to, using any of various image processing techniques with a machine learning model (e.g. a convolutional neural network (CNN) or other type of artificial neural network (ANN)) to process the density maps to select optimal entry and target points for a preliminary trajectory specific to each patient. The generated surgical plan may include at least one trajectory of the surgical tool for at least one ROI in the anatomical feature of the subject patient.

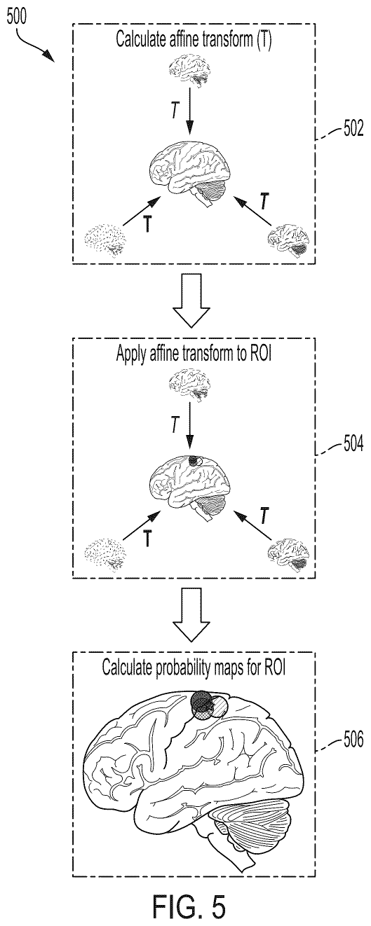

FIG. 5 is an example data flow 500 for calculating density maps for trajectory points of a surgical tool targeting at least one ROI in an anatomical template of a human brain according to some embodiments of the disclosure. Like the anatomical template 400 of FIG. 4 described above, the anatomical template in this example may represent an average of the brain images for a group of patients. While the brain images for a group of only three patients (e.g., corresponding to brain images 4101, 4102, and 4103 of FIG. 4) are shown in FIG. 5, it should be appreciated that embodiments of the present disclosure are not intended to be limited thereto and that a similar data flow may be used to calculate density maps from the registered brain images and surgical plans corresponding to any number of patients. In some embodiments, the surgical plans may also include information identifying a plurality of targeted regions in the anatomical feature for the group of patients. The surgical plans may further include coordinates of the trajectory (entry and target) points for each targeted region in a three-dimensional (3D) space. Such a 3D space may correspond to, for example, a native space representing a coordinate system and resolution of the imaging data for each patient in the group of patients. In some embodiments, one or more transforms may be calculated and applied to register the imaging data and coordinates of the respective entry and target points for each targeted region from the native space associated with each patient to a template space (e.g., a 3D template space) associated with the anatomical template, as will be described in further detail below with respect to FIG. 5.

As shown in FIG. 5, data flow 500 begins at block 502, in which a a transform is calculated to register the imaging data for each patient in the group (e.g., each of brain images 4101, 4102, and 4103) to the anatomical template. The transform may include one or more geometric operations to modify the spatial characteristics of each brain image (and image coordinates) to align with the average brain image represented by the template (and coordinate system thereof). For example, the transform for each image may be selected from a set of geometric operations that includes translations and all transformations, e.g., to scale, rotate, and/or shear the image in 3D space. In some implementations, calculating the transform may include determining the transformation parameters that best align each image or set of coordinates with the template. The determination may involve, for example, solving a mathematical optimization problem to estimate parameters that minimize the differences between the images or coordinates.

At block 504, the calculated transform from block 502 is applied to register the coordinates of the respective entry and target points for each target or targeted region in the anatomical feature to the template. In some embodiments, the transform may be applied to register or convert the coordinates of the respective entry and target points for each targeted region from a native space associated with each patient to a template space associated with the template, as described above. In some implementations, the imaging data may be spatially encoded with the coordinates of the entry and target points for each targeted region, and the transform calculated at block 502 may be applied at block 504 to register the imaging data together with the associated coordinates of the entry and target points from the native space to the template space. At block 506, the registered coordinates of the corresponding entry and target points are used to calculate one or more density maps for each targeted region. The density map(s) for each targeted region may include distributions of the corresponding entry and target points in the template space. In some embodiments, at least one of the targeted regions identified by the surgical plans in this example may be selected as the ROI at which the stereotactic procedure is to be performed.

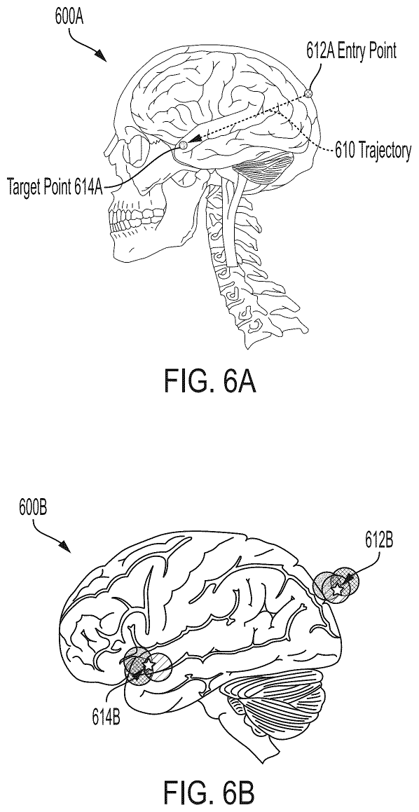

FIG. 6A is diagram of a profile view 600A of a patient's brain with an example trajectory 610 for a surgical instrument or tool between an entry point 612A and a target point 614A. The patient's brain in the example of FIGS. 6A and 6B may be based on an anatomical template representing an average of the brain images acquired for a group of patients (e.g., the average brain template 400 of FIG. 4, as described above). Entry point 612A may be a point of entry of the surgical tool into the patient's skull. Target point 614A may be a point corresponding to a ROI in the brain where the stereotactic procedure is to take place.

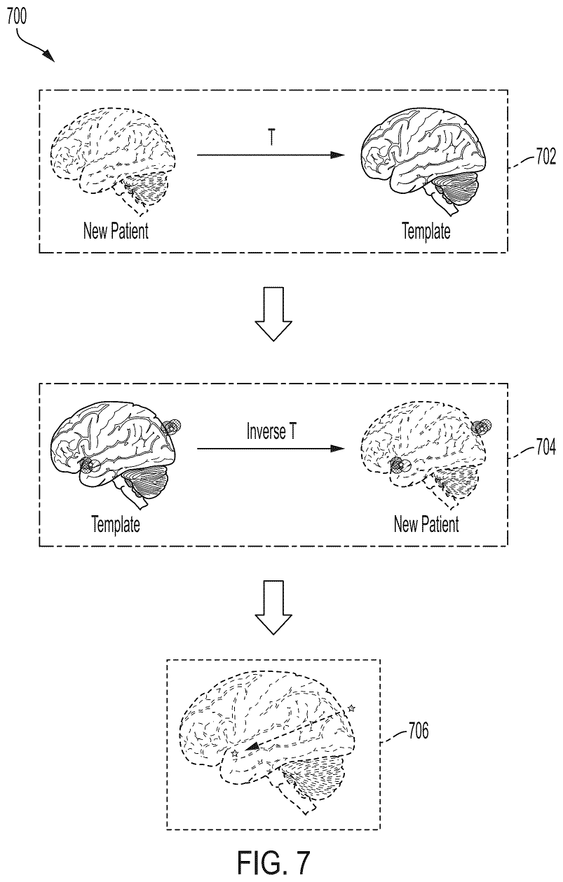

FIG. 6B is a diagram of a profile view 600B of the patient's brain with distributions 612B and 614B corresponding to the entry point 612A and the target point 614A of the trajectory 610 shown in FIG. 6A. In some embodiments, the distributions 612B and 614B may be used to calculate coordinates of the respective entry and target points 612A and 614A for the trajectory 610 of the surgical tool targeting the ROI in 3D template space. The coordinates of entry point 612A and target point 614A may correspond to, for example, a maximum density point (as represented by the star in FIG. 6B) calculated from the distributions 612B and 614B, respectively. As described above with respect to block 308 of method 300 of FIG. 3, the density maps may be used to generate a surgical plan for performing the stereotactic procedure on a new or subject patient. As will be described in further detail below with respect to FIG. 7, the surgical plan for the subject patient may be generated after first registering an image of the patient's brain to the anatomical template.

FIG. 7 is an example data flow 700 for generating a surgical plan to perform stereotactic neurosurgery on a subject patient based on density maps of entry and target points for an ROI in the patient's brain according to some embodiments of the disclosure. Data flow 700 begins at block 702, which includes calculating a transform to register an image of the anatomical feature (e.g., brain image) of the subject patient in native space to the template. The calculation at block 702 may be similar to the calculation of the transform used to register the imaging data for each patient in the group of patients (e.g., each of brain images 4101, 4102, and 4103) to the anatomical template at block 502 of FIG. 5, as described above.

At block 704, an inverse of the transform is applied to map the density maps for each respective entry and target point (in template space) to the subject's original imaging (in native space).

At block 706, a surgical plan for the subject patient is generated based on the registered image of the anatomical feature of the subject patient with the mapped coordinates of the respective entry and target points for the ROI in native space. The surgical plan includes a trajectory between the entry and target points targeting the ROI in the subject's brain.

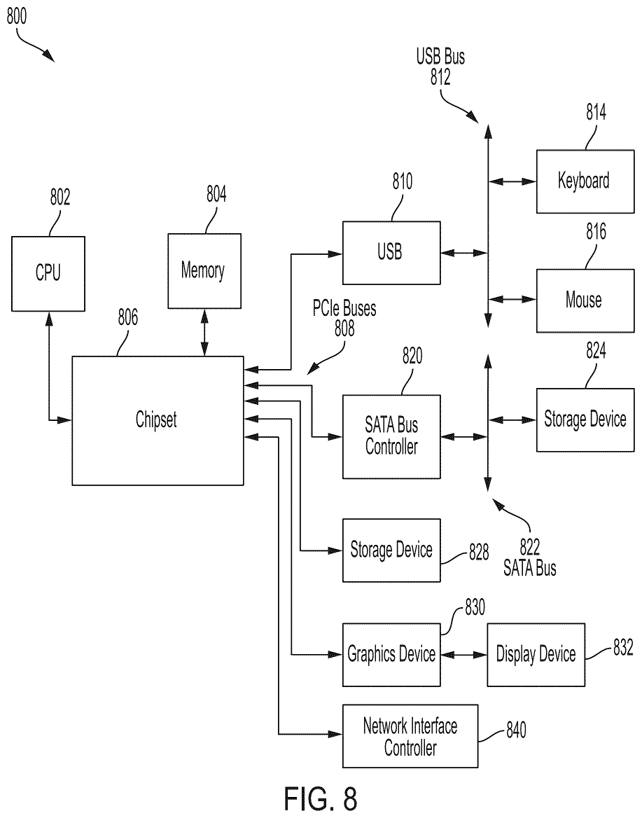

FIG. 8 is a block diagram of an example computer system 800 in which embodiments of the disclosure may be implemented. Computer system 800 may include a processor 802 (e.g., a central processing unit (CPU)), a memory (e.g., a dynamic random-access memory (DRAM)) 804, and a chipset 806. In some embodiments, one or more of the processor 802, the memory 804, and the chipset 806 may be included on a motherboard (also referred to as a mainboard), which is a printed circuit board (PCB) with embedded conductors organized as transmission lines between the processor 802, the memory 804, the chipset 806, and/or other components of the computer system. The components may be coupled to the motherboard through packaging connections such as a pin grid array (PGA), ball grid array (BGA), land grid array (LGA), surface-mount technology, and/or through-hole technology. In some embodiments, one or more of the processor 802, the memory 804, the chipset 806, and/or other components may be organized as a System on Chip (SoC).

The processor 802 may execute program code by accessing instructions loaded into memory 804 from a storage device, executing the instructions to operate on data also loaded into memory 804 from a storage device, and generate output data that is stored back into memory 804 or sent to another component. The processor 802 may include processing cores capable of implementing any of a variety of instruction set architectures (ISAs), such as the x86, POWERPC®, ARM®, SPARC®, or MIPS® ISAs, or any other suitable ISA. In multi-processor systems, each of the processors 802 may commonly, but not necessarily, implement the same ISA. In some embodiments, multiple processors may each have different configurations such as when multiple processors are present in a big-little hybrid configuration with some high-performance processing cores and some high-efficiency processing cores. The chipset 806 may facilitate the transfer of data between the processor 802, the memory 804, and other components. The chipset 806 may couple to other components through one or more PCIe buses 808.

Some components may be coupled to one or more bus lines of the PCIe buses 808. For example, components of the surgical robot 110 may be controlled through an interface coupled to the processor 802 through the PCIe buses 808. Another example component is a universal serial bus (USB) controller 810, which interfaces the chipset 806 to a USB bus 812. A USB bus 812 may couple input/output components such as a keyboard 814 and a mouse 816, but also other components such as USB flash drives, or another computer system. Another example component is a SATA bus controller 820, which couples the chipset 806 to a SATA bus 822. The SATA bus 822 may facilitate efficient transfer of data between the chipset 806 and components coupled to the chipset 806 and a storage device 824 (e.g., a hard disk drive (HDD) or solid-state disk drive (SDD)). The PCIe bus 808 may also couple the chipset 806 directly to a storage device 828 (e.g., a solid-state disk drive (SDD)). A further example of an example component is a graphics device 830 (e.g., a graphics processing unit (GPU)) for generating output to a display device 832, a network interface controller (NIC) 840 (which may provide wired or wireless access to a local area network (LAN) or a wide area network (WAN) device).

The schematic or flow diagrams of FIGS. 3, 5 and 7 are generally set forth as logical flow chart diagrams. As such, the depicted order and labeled steps are indicative of aspects of the disclosed method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Machine learning models, as described herein, may include logistic regression techniques, linear discriminant analysis, linear regression analysis, artificial neural networks, machine learning classifier algorithms, or classification/regression trees in some embodiments. In various other embodiments, machine learning systems may employ Naive Bayes predictive modeling analysis of several varieties, learning vector quantization artificial neural network algorithms, or implementation of boosting algorithms such as adaptive boosting (AdaBoost) or stochastic gradient boosting systems for iteratively updating weighting to train a machine learning classifier to determine a relationship between an influencing attribute, such as received device data, and a system, such as an environment or particular user, and/or a degree to which such an influencing attribute affects the outcome of such a system or determination of environment.

If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and certain representative advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. For example, although processors are described throughout the detailed description, aspects of the invention may be applied to the design of or implemented on different kinds of processors, such as graphics processing units (GPUs), central processing units (CPUs), and digital signal processors (DSPs). As another example, although processing of certain kinds of data may be described in example embodiments, other kinds or types of data may be processed through the methods and devices described above. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method comprising:
accessing surgical plans for previous stereotactic procedures performed on a group of patients, the surgical plans including imaging data representing an anatomical feature of each patient in the group;
applying one or more transforms to register the imaging data for each patient to a template of the anatomical feature for the group of patients;
generating one or more density maps for a trajectory targeting at least one region of interest (ROI) in the anatomical feature, based on the registered imaging data; and
generating a surgical plan to perform a current stereotactic procedure on a subject patient, based on the one or more density maps, the surgical plan including the trajectory targeting the at least one ROI in the anatomical feature of the subject patient.

2. The method of claim 1, wherein the current stereotactic procedure is a neurosurgical stereotactic procedure.

3. The method of claim 1,
wherein the surgical plans further include:
information identifying a plurality of targeted regions in the anatomical feature for the group of patients; and
coordinates of the trajectory for each targeted region of the plurality of targeted regions in a three-dimensional (3D) space, and
wherein the at least one ROI is selected from among the plurality of targeted regions.

4. The method of claim 3, wherein the trajectory includes an entry point and a target point for each targeted region of the plurality of targeted regions.

5. The method of claim 4, wherein applying the one or more transforms comprises:
calculating a first transform to register the imaging data for each patient in the group to the template of the anatomical feature; and
applying the first transform to register the coordinates of the respective entry and target points for each targeted region from a first native space associated with each patient in the group to a template space associated with the template of the anatomical feature.

6. The method of claim 5, wherein generating the surgical plan comprises:
calculating a second transform to register an image of the anatomical feature of the subject patient to the template;
applying an inverse of the second transform to map the registered coordinates of the respective entry and target points for the at least one ROI to the anatomical feature of the subject patient in a second native space; and
generating the surgical plan for the subject patient, based on the entry and target points for at least one ROI mapped to the anatomical feature of the subject patient in the second native space.

7. The method of claim 5, wherein the one or more density maps for each targeted region include distributions of the corresponding entry and target points in the template space.

8. The method of claim 7, wherein generating the one or more density maps further comprises: calculating the coordinates of the respective entry and target points for each targeted region in the template space based on the corresponding distributions.

9. The method of claim 1, wherein the current stereotactic procedure is performed using a robotic surgical tool attached to a robotic arm of a surgical robot, and where the method further comprises:
controlling the robotic arm of the surgical robot to perform the current stereotactic procedure on the subject patient according to the surgical plan.

10. The method of claim 9, wherein each of the one or more density maps includes a distribution of each trajectory point associated with the trajectory of the robotic arm in a three-dimensional (3D) template space representing the anatomical feature, and wherein controlling the robotic arm comprises:
controlling a position of the robotic arm during the current stereotactic procedure according to the distribution of each trajectory point associated with the trajectory of the robotic arm in the 3D template space.

11. A system comprising:
a processor; and
a memory coupled to the processor, the memory storing instructions that, when executed by the processor, cause the processor to perform operations including:
accessing surgical plans for previous stereotactic procedures performed on a group of patients, the surgical plans including imaging data representing an anatomical feature of each patient in the group;
applying one or more transforms to register the imaging data for each patient to a template of the anatomical feature for the group of patients;
generating one or more density maps for a trajectory targeting at least one region of interest (ROI) in the anatomical feature, based on the registered imaging data; and
generating a surgical plan to perform a current stereotactic procedure on a subject patient, based on the one or more density maps, the surgical plan including the trajectory targeting the at least one ROI in the anatomical feature of the subject patient.

12. The system of claim 11, wherein the current stereotactic procedure is a neurosurgical stereotactic procedure.

13. The system of claim 11,
wherein the surgical plans further include:
information identifying a plurality of targeted regions in the anatomical feature for the group of patients; and
coordinates of the trajectory for each targeted region of the plurality of targeted regions in a three-dimensional (3D) space, and
wherein the at least one ROI is selected from among the plurality of targeted regions.

14. The system of claim 13, wherein the trajectory includes an entry point and a target point for each targeted region of the plurality of targeted regions.

15. The system of claim 14,
wherein the operations further comprise:
calculating a first transform to register the imaging data for each patient in the group to the template of the anatomical feature; and
applying the first transform to register the coordinates of the respective entry and target points for each targeted region from a first native space associated with each patient in the group to a template space associated with the template of the anatomical feature, and
wherein the one or more density maps for each targeted region include distributions of the corresponding entry and target points in the template space.

16. The system of claim 15, wherein the operations further include:
calculating a second transform to register an image of the anatomical feature of the subject patient to the template;
applying an inverse of the second transform to map the registered coordinates of the respective entry and target points for the at least one ROI to the anatomical feature of the subject patient in a second native space; and
generating the surgical plan for the subject patient, based on the entry and target points for at least one ROI mapped to the anatomical feature of the subject patient in the second native space.

17. The system of claim 11, wherein the current stereotactic procedure is performed using a robotic surgical tool attached to a robotic arm of a surgical robot, and where the operations further include:
controlling the robotic arm of the surgical robot to perform the current stereotactic procedure on the subject patient according to the surgical plan.

18. The system of claim 17, wherein each of the one or more density maps includes a distribution of each trajectory point associated with the trajectory of the robotic arm in a three-dimensional (3D) template space representing the anatomical feature, and wherein the operations for controlling the robotic arm include:
controlling a position of the robotic arm during the current stereotactic procedure according to the distribution of each trajectory point associated with the trajectory of the robotic arm in the 3D template space.

19. A surgical robot comprising:
a robotic arm including a surgical tool for performing a stereotactic procedure on a subject patient; and
a control unit to perform a plurality of operations, the plurality of operations including:
accessing surgical plans for previous stereotactic procedures performed on a group of patients, the surgical plans including imaging data representing an anatomical feature of each patient in the group;
applying one or more transforms to register the imaging data for each patient to a template of the anatomical feature for the group of patients;
generating a density map of trajectory points for a trajectory of the robotic arm targeting at least one region of interest (ROI) in the anatomical feature, based on the registered imaging data; and
generating a surgical plan to perform a current stereotactic procedure on the subject patient, based on the density map, the surgical plan including the trajectory of the robotic arm targeting the at least one ROI in the anatomical feature of the subject patient.

20. The surgical robot of claim 19, wherein the operations further include:
controlling the robotic arm to perform the current stereotactic procedure on the subject patient according to the surgical plan.
